(19) United States
(12) Patent Application Publication
(10) Pub. No.: US 2021/0115131 A1
(43) Pub. Date: Apr. 22, 2021

(54) Treatment of Placental Chronic Histiocytic Intervillositis Using an Inhibitor of Interleukin-1

(71) Applicants: Université de Bordeaux, Bordeaux (FR); Centre National de la Recherche Scientifique, Paris (FR); Centre Hospitalier Universitaire de Bordeaux, Talence (FR)

(72) Inventors: Patrick Blanco, Verdelais (FR); Estibaliz Lazaro, Mérignac (FR); Fanny Sauvestre, Pessac (FR); Aurélien Mattuizzi, Bordeaux (FR)

(21) Appl. No.: 17/251,059

(22) PCT Filed: Jun. 13, 2019
(86) PCT No.: PCT/EP2019/065617

§ 371 Date: Dec. 10, 2020

(30) Foreign Application Priority Data
Jun. 14, 2018 (EP) 18305729.8

(51) Int. Cl. C07K 16/24; G01N 33/68

(52) U.S. Cl. CPC C07K 16/245; G01N 33/6869; A61K 2039/507; G01N 2800/368; G01N 2333/545; G01N 2800/52

(57) ABSTRACT

The present invention concerns the use of an inhibitor of interleukin-(IL-1), in particular of IL-1α and/or IL-1β, for the prevention or treatment of chronic histiocytic intervillositis (CHI) or a symptom associated thereof, eventually in combination with the use of at least one molecule conventionally prescribed to treat CHI and/or an interleukin-18 (IL-18) inhibitor. Said inhibitor of interleukin-1 (IL-1), in particular of IL-1α and/or IL-1β, may also be used for diagnosing in vitro CHI in a subject suspected of suffering from CHI or for monitoring in vitro the effectiveness of a treatment for CHI in a subject in need thereof.

### TECHNICAL FIELD OF THE INVENTION

The present invention concerns the use of an inhibitor of interleukin-1 (IL-1), in particular of IL-1α and/or IL-1 β, for the prevention or treatment of chronic histiocytic intervillositis (CHI) or an associated symptom, as well as for diagnosis or treatment monitoring.

### BACKGROUND

Among pregnancy pathologies, chronic histiocytic intervillositis (CHI) is a rare placental pathology responsible for an alteration in maternal-fetal exchanges that can lead to severe obstetrical complications (Doss et al., Hum. Pathol., 26(11): 1245-1251, 1995) [1]. Initially described by Labarrere et Mullen (1987) [2], CHI is microscopically characterized by a mononuclear cell infiltrate (about 80% macrophages, and 20% lymphocytes) and fibrinoid material deposits in the intervillous chamber. This obstetrical pathology, with a poor diagnosis (recurrent early miscarriages, severe and early in utero growth retardation, in utero fetal death), occurs with a high rate of recurrence (about 18 to 67% according to the studies) (Marchaudon et al., Placenta, 32(2): 140-145, 2011; Boyd et al., Hum. Pathol., 31(11): 1389-1396, 2000; Mekinian et al., Autoimmunity, 48(1): 40-45, 2015) [3-5]. The diagnosis of CHI is made a posteriori after anatomopathological analysis of the placenta. To date, there is no consensus on therapeutic management. Treatments usually used in vasculo-placental or immunological pathologies (e.g. aspirin, corticosteroids, low molecular weight heparins, hydroxychloroquine, immunosuppressants, polyvalent immunoglobulins, etc. . . . ) are poorly codified due to the lack of knowledge of pathophysiology, and the evaluation of their efficacy, alone or in combination, remains uncertain in the literature due to a lack of power and a lack of randomization of the studies.

There is therefore a need for a novel therapeutic protocol effective especially in the prevention or treatment of chronic histiocytic intervillositis (CHI) or associated symptoms.

### DESCRIPTION

Initially, the Inventors performed a transcriptomic immunologic analysis to identify key immune cells and pathways involved in chronic histiocytic intervillositis (CHI) pathophysiology. This analysis has been made according to Nanostring® technology which has the merit of detecting with a significant specificity partially degraded mRNAs (up to 50 bp). Indeed, because of the rarity of CHI, the only study material available is paraffin-embedded placental tissue collected for years in the hospital for which RNA extraction does not allow to obtain a satisfactory quality of RNA for transcriptomic analysis by other more conventional techniques (RTqPCR, etc. . . . ). To do this, the Inventors compared paraffinized samples of CHI placentas to paraffinized samples of healthy placentas (control), and were able to demonstrate a significant overexpression of the actors of the inflammasome activation pathway (NLRP3, NOD2, NLRC5, ASC) as well as the cytokines it produces (IL-1β and IL-18) in pathological placentas.

Based on these transcriptomic analyses, the Inventors confirmed by immunohistochemistry the overexpression of the IL-1 (in particular IL-1β) protein level in CHI placentas compared to healthy placentas (control).

From these pathophysiological data, the Inventors have demonstrated the efficacy of the inhibition of IL-1 (in particular of IL-1α and/or IL-1β) in the prevention or treatment of CHI or associated symptoms such as early miscarriage, in utero growth retardation, or in utero fetal death, wherein said symptoms are at least partly due to CHI.

The IL-1 inhibitor may be used in combination with at least one molecule conventionally prescribed to prevent or treat CHI, for instance chosen from the group consisting of aspirin, low molecular weight heparins, hydroxychloroquine, corticosteroids, azathioprine as immunosuppressant, polyvalent immunoglobulins and anti-TNF, and/or with an inhibitor of interleukin-18 (IL-18).

In the present invention, the term “IL-1 inhibitor” refers to a compound which typically decreases or neutralizes a biological activity of IL-1α (IL-1 alpha) and/or IL-1β (IL-1 beta). This inhibitor is preferably a direct inhibitor of IL-1α and/or IL-1β. This means that the inhibitor typically directly decreases or neutralizes a biological activity of IL-1 or its receptor, or in other words, that the IL-1 decreased or neutralized biological activity is not the result of the action of an intermediate compound other than IL-1 receptor.

Examples of IL-1 inhibitors of interest are antibodies directed against IL-1α and/or IL-1β and/or IL-1 receptor, aptamers or spiegelmers directed against IL-1α and/or IL-1β and/or IL-1 receptor, inhibitory nucleic acid sequences directed against IL-1α and/or IL-1β and/or IL-1 receptor, IL-1 receptor antagonists, and small molecules directed against IL-1α and/or IL-1β and/or IL-1 receptor.

According to particular embodiments of the present invention, the IL-1 inhibitor specifically inhibits IL-1α, specifically IL-1β, specifically inhibits IL-1 receptor, or specifically inhibits both IL-1α and IL-1β.

The present invention thus concerns an inhibitor of IL-1α and/or IL-1β for use in the prevention or treatment of CHI or at least one associated symptom such as early miscarriage, in utero growth retardation, or in utero fetal death, wherein said symptom is at least partly due to CHI.

The present invention also concerns a pharmaceutical composition comprising an inhibitor of IL-1α and/or IL-1β for use in the prevention or treatment of CHI or at least one associated symptom such as early miscarriage, in utero growth retardation, or in utero fetal death, wherein said symptom is at least partly due to CHI. The pharmaceutical composition for use according to the invention further comprises at least one pharmaceutically acceptable excipient, vehicle, carrier or support in addition to the IL-1 inhibitor. The term “pharmaceutically acceptable” means approved by a regulatory agency of the Federal or a state government or listed in the U.S. Pharmacopeia, or European Pharmacopeia or other generally recognized pharmacopeia for use in animals, and more particularly in humans.

A typical IL-1 inhibitor for use according to the present invention, or herein described composition comprising such an inhibitor for use according to the present invention, may be administered to a subject by way of injection in the bloodstream (systemic injection), such as intra-venous or intra-arterial injection, by way of subcutaneous or cutaneous administration, by way of intramuscular administration, by way of oral (per os) or rectal administration, and/or by way of nasal administration. In a preferred embodiment, the composition is formulated for subcutaneous administration.

The inhibitor of IL-1α and/or IL-1β for use according to the invention or comprised in the pharmaceutical composition for use according to the invention is preferably selected from the group consisting of canakinumab (Ilaris®, a human monoclonal antibody of IgG1/kappa isotype targeted at interleukin-1 beta) and anakinra (Kineret®, an interleukin-1 receptor antagonist).

According to a particular embodiment of the present invention, said pharmaceutical composition for use according to the invention further comprises at least one molecule conventionally prescribed to treat CHI preferably chosen from the group consisting of aspirin, low molecular weight heparins, hydroxychloroquine, corticosteroids, azathioprine as immunosuppressant, polyvalent immunoglobulins and anti-TNF compounds. Said molecule when present is administered together, concomitantly or sequentially with the inhibitor of IL-1α and/or IL-1β.

According to a particular embodiment of the present invention, said pharmaceutical composition for use according to the invention further comprises at least one interleukine 18 (IL-18) inhibitor. As example of IL-18 inhibitor can be cited the recombinant human IL-18 binding protein known as tadekinig alpha. According to a particular embodiment of the present invention, said IL-18 inhibitor is an anti-IL-18 antibody. Said IL-18 inhibitor when present is administered together, concomitantly or sequentially with the inhibitor of IL-1α and/or IL-1β.

According to a particular embodiment of the present invention, said inhibitor of IL-1α and/or IL-1β is an anti-IL-1 antibody, preferably an anti-IL-1β antibody. For example, the anti-IL-1β monoclonal antibody Canakinumab (Ilaris®) can be used in the prevention or treatment of CHI or associated symptoms such as early miscarriage, in utero growth retardation, or in utero fetal death as inhibitor of IL-1α and/or IL-1β.

According to a particular embodiment of the present invention, said inhibitor of IL-1α and/or IL-1β is an antagonist of IL-1 receptor. For example, the biopharmaceutical drug anakinra (Kineret®) which is able to inhibit both IL-1α and IL-1β can be used in the prevention or treatment of CHI or associated symptoms such as early miscarriage, in utero growth retardation, or in utero fetal death as inhibitor of IL-1α and/or IL-1.

According to a particular embodiment of the present invention, said inhibitor of IL-1α and/or IL-1β is used at conventional doses for said compound, for instance at a posology of 100 mg/day, in particular a posology of 100 mg/day subcutaneously, preferably in one single administration per day. Doses may vary depending on the route of administration and/or the optional presence of an additional biologically active compound.

A further object of the invention is a method for diagnosing in vitro CHI in a subject comprising the steps of:
a) Measure of the expression level E1 of IL-1α and/or IL-1β, preferably IL-1, in a sample of a subject;
b) Comparison of the expression level E1 to a reference expression level E2 of IL-1α and/or IL-1β, preferably IL-1β, in a sample of a healthy subject, wherein a value E1 greater for IL-1α and/or IL-1, preferably IL-1β, than a value E2 is the indication of a subject suffering from CHI.

According to the present invention, step a) of the method for diagnosing in vitro CHI in a sample of a subject suspected of suffering from CHI may be performed by any appropriate technique known in the art to measure the expression level of IL-1α and/or IL-1β, preferably IL-1 β. Step a) may be performed by measuring the expression of the IL-1, preferably IL-1α and/or IL-1β protein, or the level of IL-1α and/or IL-1β, preferably IL-1β, mRNA. When the expression level of IL-1α and/or IL-1β protein, preferably IL-1β, is used, the sample is preferably a blood sample or a placenta sample, in particular a blood sample. When the expression level of IL-1α and/or IL-1β mRNA, preferably IL-1 β mRNA, is used, the sample is preferably a placenta sample. For comparison purposes, samples from a subject to be diagnosed and from a healthy subject are similar in nature so that their expression levels are directly comparable. For instance, step a) may be performed by ELISA, qPCR or by Nanostring analysis.

In the present invention, the expression “reference expression level [ . . . ] in the sample of a healthy subject” refers to a reference subject or group of reference subjects not presenting and/or having not presented a CHI state or symptoms associated thereof. In the present invention, the term “group of reference subjects” refers to a group making it possible to define a reliable reference value. It may, for example, a group of at least 2 reference subjects as defined above, of at least 40 reference subjects, or of at least 40 to 200 reference subjects. The reference subject or group of reference subjects preferably has similar physiological characteristics as the subject to be diagnosed for CHI, e.g. chosen in a group including similar age, weight, sex, body mass, and/or tobacco/alcohol/drug abuse.

A further object of the invention is a method for monitoring in vitro the effectiveness of a treatment for CHI in a subject, comprising the steps of:
a′) Measure of the expression level E1′ of IL-1α and/or IL-1β, preferably IL-1β, in a sample of a subject suffering from CHI before receiving any treatment of CHI;
b′) Measure of the expression level E2′ of IL-1α and/or IL-1β, preferably IL-1β, in a sample of the subject suffering from CHI after beginning treatment of CHI;
c′) Comparison of expression levels E1′ and E2′, wherein a value E2′ less than a value E1′ is the indication of an effective treatment.

According to the present invention, steps a′) and b′) of the method for monitoring in vitro the effectiveness of a treatment for CHI in a sample of a subject suffering from CHI may be performed by any appropriate technique known in the art to measure the expression level of IL-1α and/or IL-1β, preferably IL-1β. Step a) may be performed by measuring the expression of the IL-1, preferably IL-1α and/or IL-1β protein, or the level of IL-1α and/or IL-1β, preferably IL-1β, mRNA. When the expression level of IL-1α and/or IL-1β protein, preferably IL-1β, is used, the sample is preferably a blood sample or a placenta sample, in particular a blood sample. When the expression level of IL-1α and/or IL-1β mRNA, preferably IL-1β mRNA, is used, the sample is preferably a placenta sample. For comparison purposes, samples derived at different times from a subject suffering from CHI are similar in nature so that their expression levels are directly comparable. For instance, steps a′) and b′) may be performed by ELISA, qPCR or by Nanostring analysis.

In the present invention, the term “treatment” refers to a medical treatment, for example allopathic, involving the taking of molecules (e.g. chemical molecules, for example molecules obtained by organic synthesis, molecules of biological origin, for example proteins, molecules from living organisms, for example mammals, microorganisms, plants and/or synthesized by living organisms, for example proteins, nucleic acid molecules). The term “treatment” refers to therapeutic intervention in an attempt to alter the natural course of the subject being treated. Desirable effects of treatment include, but are not limited to, attenuation or alleviation of symptoms, diminishment of any direct or indirect pathological consequences of CHI or associated symptoms as disclosed herein, decreasing the rate of CHI or associated symptoms as disclosed herein progression, and amelioration or palliation of the CHI or associated symptoms as disclosed herein state.

In the present invention, the term “prevention” refers to a treatment performed for preventive (prophylactic) purpose. Desirable effects of prevention include, but are not limited to, preventing occurrence or recurrence of CHI or associated symptoms as disclosed herein.

In the present invention, the treatment of CHI may be any treatment known to those skilled in the art. According to the present invention, this may be, for example, a treatment using an inhibitor of IL-1α and/or IL-1β. This may be, for example, a further treatment using one molecule conventionally prescribed to treat CHI preferably chosen from the group consisting of aspirin, low molecular weight heparins, hydroxychloroquine, corticosteroids, azathioprine as immunosuppressant, polyvalent immunoglobulins and anti-TNF compounds. This may be, for example, a further treatment using an inhibitor of IL-18.

### BRIEF DESCRIPTION OF THE FIGURES

FIG. 1 represents violin plot of differential expression levels of proteins involved in the inflammasome pathway (CASP1, PYCARD, NOD2, NLRP3) of healthy placentas (control, CTRL) versus those with CHI (intervillitis, CHI) (*: p<0.05; **: p<0.01).

FIG. 2 represents violin plot of differential expression levels of IL-1β and IL-18 mRNA of healthy placentas (control, CTRL) versus those with CHI (intervillitis) (*: p<0.05; **: p<0.01).

FIG. 3 represents (A) the immunohistochemical labelling of the IL-1β protein in tissue sections of CHI placentas (intervillitis, CHI) compared to samples of healthy placentas (control, CTRL) (B) the number of IL-1β R clusters in tissue sections of CHI placentas (intervillitis, CHI) compared to samples of healthy placentas (control, CTRL).

### EXAMPLES

### Example 1: Differential Expression of mRNA of the Inflammasome Activation Pathway of CHI Placentas Compared to Healthy Placentas

In the context of the present invention, RNA from 18 paraffinized samples of CHI placentas (intervillitis) grade II or III and 6 paraffinized samples of healthy placentas (control) was extracted with Qiagen RNeasy FFPE kit. For each patients, two 20 μm placenta tissue sections were used. After the deparaffinization step, tissues were digested with proteinase K. Supernatants were treated with DNAse I. Total RNA were then treated trough RNeasy MinElute column and eluted with 30 μl of RNase-free water.

The transcriptomic analysis of the 24 patients samples was made according to the manufacturer Nanostring protocol by using the PanCancer Immune Profiling Pattern. The samples were analysed with the nCounter XT Gene Expression Assays leading to the mRNA counts for each sample.

The statistical analysis was made with the nSolver Advanced analysis software. Four different plug-ins were used to analyse the data: plug-in Overview, plug-in Normalization, plug-in Gene set analysis and plug-in Cell type profiling.

The results obtained are shown in the table 1 below.

TABLE 1

| | Differential | | p. adjusted |
|---|---|---|---|
| rRNAm | expression | Fold change | value |
| NOD2 (NLRC2) | yes | 6.21 | 0.00705 |
| NLRC5 | yes | 4.54 | 0.0218 |
| NLRP3 | yes | 3.14 | 0.0422 |
| PYCARD (ASC) | yes | 5.68 | 0.0088 |

The results showed a significative overexpression of of the actors of the inflammasome activation pathway: PYCARD (Fold Change=5.58: 0.01), NLRP3 (Fold Change=3.14; p<0.05), NLRC5 (Fold Change=4.54; p<0.05) et NOD2 (Fold Change=6.21; p<0.01) in pathological placentas compared to healthy placentas.

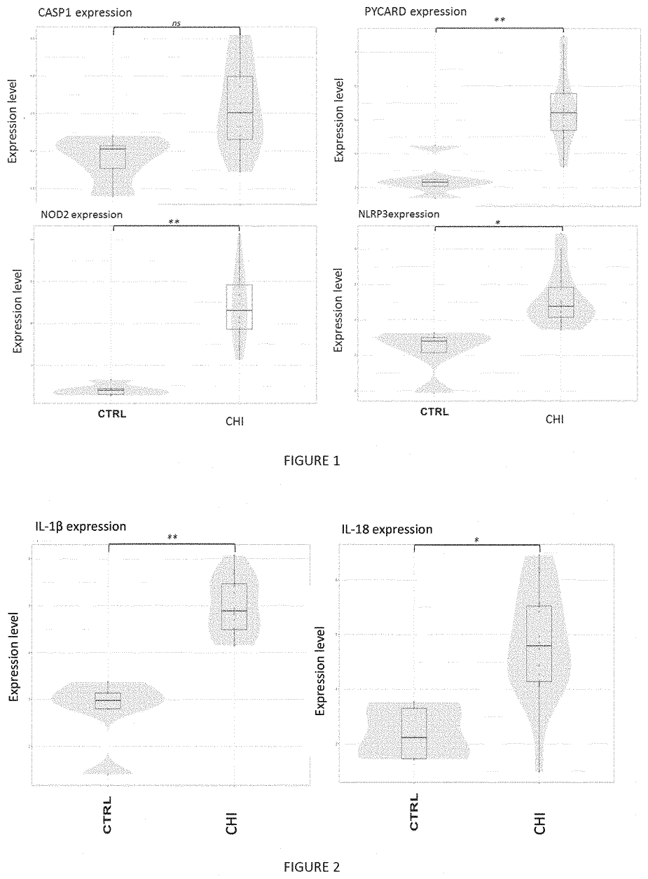

The significant overexpressions of PYCARD, NOD2 and NLRP3 mRNA were represented in violin plots in FIG. 1.

### Example 2: Expression Level of IL-1β and IL-18 mRNA of CHI Placentas Compared to Healthy Placentas

In the context of the present invention, RNA from 18 paraffinized samples of CHI placentas (intervillitis) grade II or III and 6 paraffinized samples of healthy placentas (control) was extracted with Qiagen RNeasy FFPE kit. For each patients, two 20 μm placenta tissue sections were used. After the deparaffinization step, tissues were digested with proteinase K. Supernatants were treated with DNAse I. Total RNA were then treated trough RNeasy MinElute column and eluted with 30 μl of RNase-free water.

The transcriptomic analysis of the 24 patients samples was made according to the manufacturer Nanostring protocol by using the PanCancer Immune Profiling Pattern. The samples were analysed with the nCounter XT Gene Expression Assays leading to the mRNA counts for each sample.

The statistical analysis was made with the nSolver Advanced analysis software. Four different plug-ins were used to analyse the data: plug-in Overview, plug-in Normalization, plug-in Gene set analysis and plug-in Cell type profiling.

The results obtained are shown in the table 2 below.

TABLE 2

| | Differential | | p. adjusted |
|---|---|---|---|
| rRNAm | expression | Fold change | value |
| IL-1β | yes | 5.66 | 0.00498 |
| IL-18 | yes | 3.96 | 0.0343 |

The results showed a significative overexpression of the cytokines produced by the inflammasome actors in pathological placentas: IL-1β (Fold change=5.66; p<0.01) and 11-18 (Fold Change=3.96; p<0.05).

The significant overexpressions of IL-1β and IL-18 mRNA were represented in violin plots in FIG. 2.

### Example 3: Overexpression Level of IL-1β Protein in CHI Placentas Compared to Healthy Placentas

In the context of the present invention, the IL-1β protein expression level was analysed by immunohistochemistry.

All samples have been fixed in 10% buffered formalin. Immunohistochemical staining was performed for all samples with anti-IL1β antibody (Cell Signaling®, clone 3A6, dilution 1/100).

Formalin-fixed and paraffin-embedded tissues were cut (3 μm thickness) and mounted onto slides. Specimens were deparaffinated (OTTIX) for 6 min. After rinsing in distilled water, slides were incubated at 95-100° C. for 20 min with unmasking solution. After rinsing in TBS-Tween (0.1%), endogenous peroxidase activity was blocked by 3% H₂O₂ for 10 min at room temperature. Blocking buffer was added onto the sections of the slides in a humidified chamber at room temperature for 20 min. Then, 100 μl diluted primary antibody were added to the sections on the slides and incubated overnight in a humidified chamber at 4° C. After rinsing, 100 μl appropriately diluted secondary antibody were added to the sections, slides were incubated in a humidified chamber at room temperature for 30 min. After washing, 100 μl DAB substrate solution were applied. Slides were counterstained with hematoxylin and mounted. Immunohistochemically stained slides were reviewed without knowledge of the diagnosis. Numbers of aggregates were evaluated in 50 high power fields for each sample.

The labelling was made according to the ImmPress manufacturer protocol.

The antibodies used are described in table 3.

TABLE 3

| | | | | Secondary |
|---|---|---|---|---|
| Target | Provider | Reference | Dilution | Antibody |
| IL-1β | Cell signaling | 3A6 | 1/100 | mouse |
| Mouse | VectorLab | ImmPRESS | — | — |
| secondary | | HRP Reagent | | |
| antibody | | kit | | |
| | | MP-7402 | | |

The labelling was analysed in a blinding way by an anatomopathologist specialized in placenta analysis.

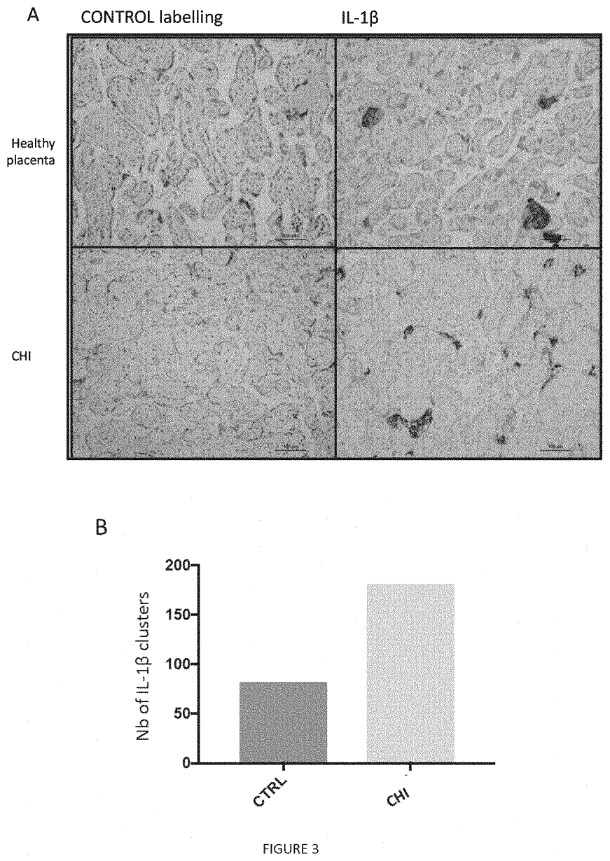

The labelling results are shown in FIG. 3A. The results showed that the IL-1β protein is localized in the syncytial knots and in the fibrinoid substance deposits.

The quantitative analysis showed in FIG. 3B that the IL-1β protein is overexpressed in CHI placentas (180 IL-1β clusters counted for fifty high power fields analysed) compared to healthy placentas (81 IL-1β clusters counted for fifty high power fields analysed)

### Example 4: Evaluation of the Interest of the Biopharmaceutical Drug Kineret® (Anakinra) in the Prevention of Recurrence of Chronic Histiocytic Intervillitis

This is a phase I/II pilot clinical trial regarding recurrent CHI. Although this is a rare disease, patients with recurrent CHI are frequently sent to referral centers due to the therapeutic impasse and are seeking new therapeutic strategies.

Although data on anakinra during pregnancy are limited, no effect of anakinra has been reported in terms of fertility, embryo-fetal, peri- or post-natal development (rats and rabbits) at supra-therapeutic doses. The literature provides reassuring data on the tolerance of this molecule in humans during pregnancy, for conditions for which anakinra cannot be suspended during pregnancy (Still's disease, periodic syndrome associated with cryopyrin).

The main objective of this study is therefore to demonstrate that ankinra can be a promising therapy for the prevention of recurrent CHI, but secondary purposes are also to evaluate the effect of anakinra during pregnancy in terms of: obstetrical outcome, clinical-biological tolerance of a the molecule during pregnancy and within 12 months postpartum, and impact of the treatment on placental anatomo-pathology.

The cohort studied comprises 15 patients with a history of recurrent CHI: at least two documented episodes of CHI leading to obstetric complications (spontaneous miscarriage, late fetal loss, intrauterine growth retardation, fetal death in utero, pre-eclampsia). CHI is diagnosed during histological examination of the placenta of the previous pregnancy according to the classification of Rota et al. (J. Gynecol. Obstet. Biol. Reprod., 35(7): 711-719, 2006) [6]. It is to be noted that minor patients, patients unable to give informed consent, patients having an immunosuppressive treatment during pregnancy, patients with immune deficiency and patients with known hypersensitivity to anakinra, are excluded from the study. This inclusion period of patients lasts 24 months.

Patients randomized to the anakinra group receive anakinra at a dose of 100 mg/day subcutaneously once daily from the beginning of pregnancy (no later than 8 weeks of gestation) and up to 34 weeks of amenorrhea. This participation period lasts in fact 21 months (duration of pregnancy and 12 months of post-partum follow-up).

The pregnancy outcome is evaluated according to a main judgment criterion based on the live birth rate for these patients at high risk of recurrence of CHI (rate which may exceed 60%), as well as to secondary judgment criteria based on obstetrical complications (spontaneous miscarriages, vasculoplacental unsufficiency, fetal death in utero, prematurity), maternal infectious complications during pregnancy and during post-partum, fetal malformations, pediatric infectious complications in the first year of life, and histogical examination of the placenta and CHI grade evaluation if present.

### LIST OF REFERENCES

1. Doss et al., Hum. Pathol., 26(11): 1245-1251, 1995
2. Labarrere et Mullen, Am. J. Reprod. Immunol. Microbiol., 15(3): 85-91, 1987
3. Marchaudon et al., Placenta, 32(2): 140-145, 2011
4. Boyd et al., Hum. Pathol., 31(11): 1389-1396, 2000
5. Mekinian et al., Autoimmunity, 48(1): 40-45, 2015
6. Rota et al., J. Gynecol. Obstet. Biol. Reprod., 35(7): 711-719, 2006

1. A method of treating chronic histiocytic intervillositus (CHI) or at least one associated symptom, comprising administering an inhibitor of IL-1α and/or IL-1ß, wherein said symptom is at least partly due to CHI.

2. The method according to claim 1, wherein said inhibitor is an anti-IL-1 antibody.

3. The method according to claim 2, wherein said anti-IL-1 antibody is an anti-IL-1ß antibody.

4. The method according to claim 3, wherein said anti-IL-1ß antibody is Canakinumab.

5. The method according to claim 1, wherein said inhibitor is an antagonist of IL-1 receptor.

6. The method according to claim 3, wherein said antagonist of IL-1 receptor is anakinra.

7. The method according to claim 1, wherein the associated symptom is selected from the group consisting of early miscarriage, in utero growth retardation, and in utero fetal death.

8. (canceled)

9. (canceled)

10. The method to claim 1, wherein the inhibitor of IL-1α and/or IL-1ß is administered together, concomitantly or sequentially with a molecule selected from the group consisting of aspirin, low molecular weight heparins, hydroxychloroquine, a corticosteroid, azathioprine, a polyvalent immunoglobulins, and an anti-TNF compounds.

11. The method according to claim 1, wherein the inhibitor of IL-1α and/or IL-1ß is administered together, concomitantly or sequentially with an inhibitor of interleukin-18 (IL-18).

12. The method according to claim 11, wherein the inhibitor of IL-18 is an anti-IL-18 antibody.

13. A method for diagnosing in vitro CHI in a subject comprising the steps of:
a. measuring an expression level E1 of IL-1α and/or IL-1ß in a sample subject; and
b. comparing of the expression level E1 to a reference expression level E2 of IL-1α and/or IL-1ß, in a sample from a healthy subject, wherein a value E1 greater for IL-1α and/or IL-1ß than a value E2 is the indication of a subject suffering from CHI.

14. A method for monitoring in vitro the effectiveness of a treatment for CHI in a subject, comprising the steps of:
a. measuring an expression level E1′ of IL-1α and/or IL-1ß in a sample of a subject suffering from CHI before the subject receiving any treatment for CHI;
b. measuring an expression level E2′ of IL-1α and/or IL-1ß in a sample of the subject suffering from CHI after beginning treatment for CHI; and
c. comparing expression levels E1′ and E2′, wherein a value E2′ less than a value E1′ indicates effective treatment.
